(19) United States
(12) Plant Patent Application Publication
(10) Pub. No.: US 2002/0124287 P1
(43) Pub. Date: Sep. 5, 2002

(54) Cooking banana plant "FHIA-25"

(76) Inventor: Phillip Ray Rowe, La Lima (HN)

Correspondence Address: GARVEY SMITH NEHRBASS & DOODY, LLC, THREE LAKEWAY CENTER, 3838 NORTH CAUSEWAY BLVD., SUITE 3290, METAIRIE, LA, 70002

(21) Appl. No.: 09/799,208

(22) Filed: Mar. 5, 2001

(51) Int. Cl. A01H 5/00

(52) U.S. Cl. PLT/160

(57) ABSTRACT

This new and distinct variety of cooking banana plant has both Musa acuminata and Musa balbisiana in its pedigree. Its Latin name is designated as Musa (AAB), with A representing a genome of M. acuminata and B representing a genome of M balbisiana. It has the following unique combination of desirable features:

1. A high level of resistance to the black Sigatoka leaf spot disease (Mycosphaerella fijiensis).

2. A dwarf plant height which enables it to withstand strong winds.

3. A strong root system which makes it a hardy plant under marginal water and soil conditions.

4. Large bunch sizes which are frequently greater than 45.0 kg.

5. Green fruit has a very good flavor and texture when boiled (as thick slices) or fried (as thin slices).

6. Green fruit is easy to peel and exudes very little latex when peeled.

7. Harvested green fruit has a long green life.

### BACKGROUND OF THE INVENTION

This new variety was developed at the Fundaci6n Hondureña de Investigación Agricola (FHIA) in La Lima, Honduras from a cross made in 1995. It was selected in 1997 from several first-generation seedlings from the cross SH-3648× SH-3142 (both unpatented and both developed from accessions and breeding lines which are unpatented and are in the FHIA germplasm collection). SH-3648, which was developed by inventor, is a dwarf bred tetraploid which is resistant to the black Sigatoka leaf spot disease. The pedigree of SH-3648 is II-408×I-63→ SH-2952× SH-2741→ SH-3386× SH-3362→ SH-3648. The two original II-408 and I-63 parental lines are the Gaddatu (ABB) triploid clone from the Philippines and a Musa balbisiana (BB) diploid clone of unknown origin which was collected in Costa Rica, respectively. SH-2741 is a dwarf bred diploid which was derived from AVP-45× AVP-23→ SH-90× II-334 SH-2518× II-158 SH-2741. The AVP-45, AVP-23, II-334 and II-158 parental lines are a wild Musa acuminata subsp. zebrina diploid accession from Java, a Robusta Cavendish triploid clone that was collected in Jamaica, a wild Musa acuminata subsp. siamea diploid from Malaysia, and a parthenocarpic Musa acuminata subsp. errans diploid from North Borneo, respectively. The SH-3362 diploid parental line of SH-3648 was selected from a segregating population derived from SH-3142× SH-3217. The SH-3142 diploid, which was developed by inventor and is the female parent of SH-3362 and the male parent of FHIA-25, was derived by crossing the SH-1734 bred diploid onto the Pisang Jari Buaya natural diploid that was collected from Papua New Guinea. The diploids which are in the pedigree of SH-1734 are the Lidi and Sinwobogi parthenocarpic clones and a Musa acuminata subsp. errans wild type that were collected in Sumatra, Irian Jaya, and the Philippines, respectively. The SH-3217 parent of SH-3362 is a complex bred diploid which has in its pedigree the parthenocarpic Guyod, Tjau Lagada, and Sinwobogi natural diploids and a wild Musa acuminata subsp. malaccensis accession from the Philippines, Java, Irian Jaya, and Malaysia, respectively. FHIA-25 was selected as a triploid hybrid which maintained the dwarf plant stature and black Sigatoka resistance of its SH-3648 tetraploid parental line, but differs from SH-3648 in that it does not produce pollen. This absence of pollen results in seedless fruit, as compared to the seedy fruit of SH-3648, and this characteristic of FHIA-25 permits it to be cultivated as a commercial variety without concerns about seediness. FHIA-25 is also readily distinguished from SH-3648 by its rounded fruit shape, which closely resembles that of the reference Cavendish export banana, as compared to a more angular fruit shape for SH-3648.

This new cooking banana plant was asexually reproduced by corms as performed by inventor in the experimental farm of FHIA in La Lima, Cortés, Honduras and shows. that all plant and fruit characteristics run true to the original selected plant and are identical in all respects.

### BRIEF SUMMARY OF THE INVENTION

This new and distinct variety of cooking banana is a vigorous dwarf plant which produces large bunches of fruit. It is highly resistant to the black Sigatoka leaf spot disease, which is the most destructive and most costly disease of bananas and plantains world-wide. Interest in this new variety is that it is the first dwarf cooking banana which is highly resistant to black Sigatoka and has excellent cooking qualities as green fruit. This combination of exceptional plant and fruit characteristics make it a candidate for cultivation as a green cooking banana in areas where black Sigatoka has severely reduced the yields of the natural banana and plantain varieties which have traditionally been cultivated for production of green fruit for cooking.

### BRIEF DESCRIPTION OF THE PHOTOGRAPHS

The accompanying photographic reproductions show typical specimens of the new cooking banana variety.

FIG. 1 shows a typical FHIA-25 Cooking Banana plant and bunch one month before the fruit is ready for harvesting.

FIG. 2 shows representative whole fingers, a longitudinal section, and cross-sections of fruit at harvest stage of the new variety (marker is 20 cm).

FIG. 3 shows a typical bunch of the FHIA-25 Cooking Banana (center) as compared to bunches of the universally cultivated False Horn plantain variety (on both sides). These bunches are from plants which received no treatment for control of black Sigatoka, and this comparison is made because it is anticipated that one of the main uses of FHIA-25 will be to replace False Horn in areas where black Sigatoka has severely reduced the yields of this plantain variety.

FIG. 4 shows detached newly formed male buds with the outer bract removed before it lifts naturally to depict botanically distinguishing size, shape and color differences in these plant parts of the Grand Nain Cavendish export banana variety (left) as compared to FHIA-25. Inside surface color of this outer bract for the new variety conforms to Plate 7-L-6.

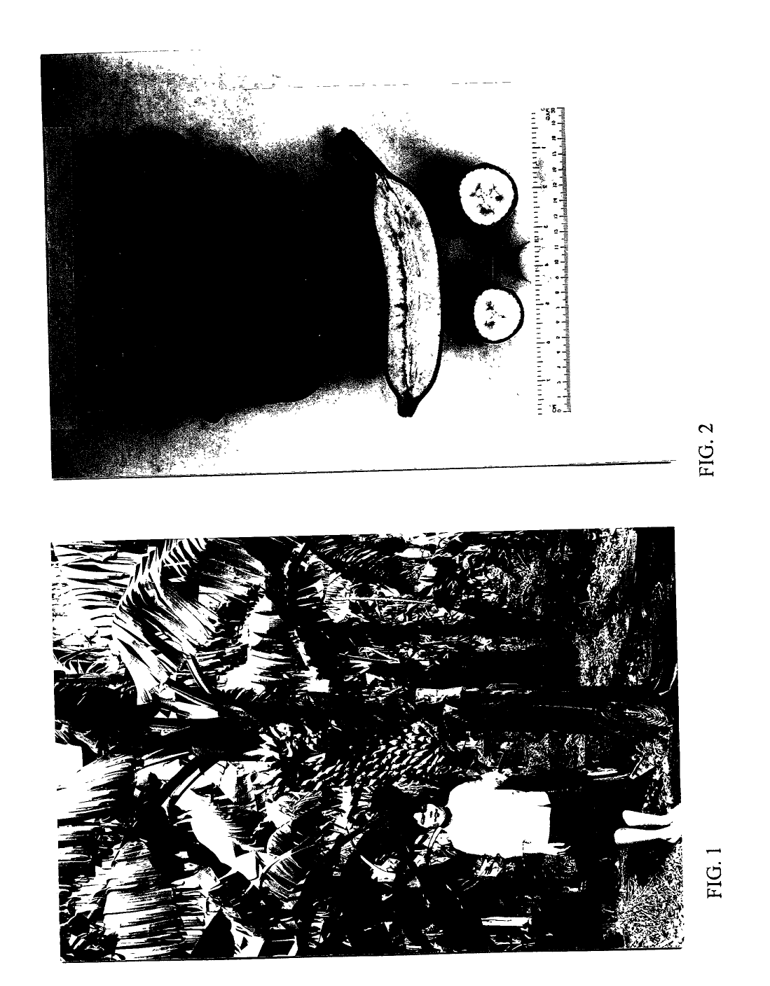

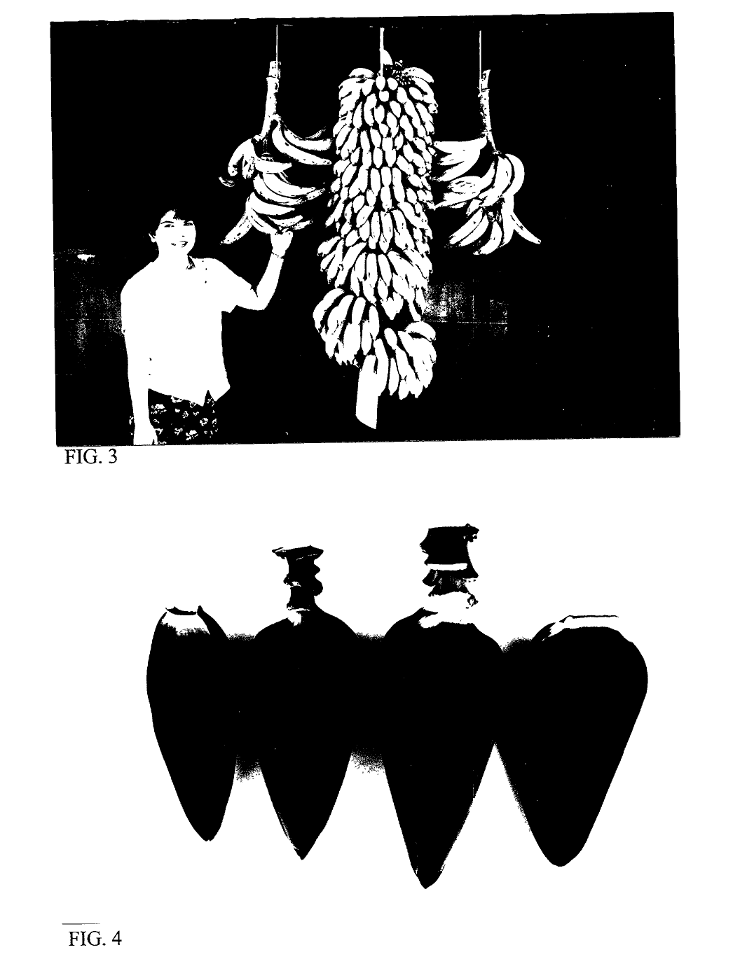

The photographs were taken on fresh material and the colors are as nearly true as is reasonably possible in color representations of this type.

### DETAILED BOTANICAL DESCRIPTION

The following is a detailed botanical description of the new and distinct variety of cooking banana plant, its flowers, foliage and fruit as based on observations of specimens grown in the FHIA experimental farm near La Lima, Cortés, Honduras. These descriptions are in accordance with the internationally standardized “Descriptor List for Bananas” elaborated by the Institut de Recherche sur les Fruits et Agrumes in Montpellier, France. Color terminology for the male bud and bracts, which have very distinctive colors for variety identification, is in accordance with the Maerz and Paul Dictionary of Color. Other colors are given in common terms for bananas.

Vegetative Characteristics (General Growth/Psedudostems/Suckers):

Ploidy.—Triploid (AAB).

General appearance.—Normal.

Characteristics of dwarfism.—Yes.

Size.—Small.

Stature.—Robust.

Color of Pseudostem.—Green/pink.

Appearance ofpseudostem.—Shiny.

Predominant color at the inside base of outer leaf sheath.—Light green

Pigmentation of internal leaf sheaths.—Purple.

Color of the sap.—Watery.

Wax on leaf sheaths.—Little.

Number ofsuckers (>30 cm).—From 3 to 5.

Development of suckers.—No inhibition

Position of growth of suckers.—Vertical growth

Vegetative Characteristics (Petioles/Midribs/Leaves):

Blotches at the base of the petiole.—Extensive pigmentation

Color of blotches.—Dark brown.

Petiole canal of leaf six.—Open with margins spreading.

Petiole margins.—Winged and clasping the pseudostem

Type of margins.—Dry.

Line of color along edge ofpetiole margin.—Yes.

Color ofpetiole margin.—Pink-red.

Width ofpetiole margin.—Wide.

Length of leafsix.—Medium (170-220 cm).

Width of leaf six.—Medium (70-80 cm).

Length of petiole.—Medium (50-70 cm).

Color of upper surface of leaf.—Green.

Appearance of upper surface of leaf.—Dull.

Color of lower surface of leaf.—Medium green.

Appearance of lower surface of leaf.—Dull.

Presence of wax on leaves.—None.

Insertion of leaf.—±Symmetric.

Base of leaf.—One side rounded.

Corrugation of leaf.—Medium corrugation

Color of dorsal face of midrib.—Pinkish tint.

Color of ventralface of midrib.—Light green.

Color of dorsalface of cigar leaf.—Reddish-purple tint.

Leaves ofyoung suckers.—Without blotches.

Inflorescence (General Appearance/Male Bud):

Length ofpeduncle.—Medium.

Number of empty nodes on peduncle.—One.

Size ofpeduncle.—Medium.

Color of peduncle.—Medium green.

Hairiness ofpeduncle.—Glabrous.

Position of bunch.13 Hanging vertically.

Shape of bunch.—Cylindrical.

Appearance of bunch.—Compact.

Flowers forming the fruit.—Female.

Fingers of the bunch.—Biseriate.

Position of rachis.—At an angle.

Appearance of rachis.—Bare.

Presence of male bud.—Present.

Shape of male bud.—Intermediate.

Size of male bud.—Large.

Bracts:

Shape of the base of the bract.—Medium.

Shape of apex of bract.—Intermediate.

Imbrication of the bract.—Young bracts slightly overlap.

Color of the externalface of the bract.—Purple (Plate 48-J-12).

Color of the internalface of the naturally lifted bract.—Red/crimson (Plate 7-L-7).

Color of the apex of the bract.—Tinted with yellow.

Stripes of color on bract.—No.

Bract scars on rachis.—Very prominent

Fading of color at the base of the inside of the bract.—Color discontinuing towards the base.

Shape of the male bract.—width/length greater than 0.30.

Lifting of male bracts.—Lifting one or two at a time.

Behavior of bracts.—Revolute.

Presence of wax on the bract.—Average wax

Presence of grooves on the bract.—Intermediate.

Male Flowers:

Behavior of male flowers.—Falling after the bract.

Basic color of compound tepal.—Cream.

Pigmentation of compound tepal.—Absent.

Color of the lobes of the compound tepal.—Yellow.

Development of the lobes of the compound tepal.—Very developed.

Color of the free tepal.—Translucent white.

Shape of the free tepal.—Oval.

Appearance of free tepal.—More or less smooth

Apex of the free tepal.—No or little development.

Shape of the apex offree tepal.—Thread-like.

Length of anthers.—Greater than compound tepal.

Color of filament.—Cream.

Color of anther (dorsal face).—Cream.

Pollen.—Absent.

Basic color of style.—Cream.

Purple pigmentation of style.—Present.

Length of style .—Longer than compound tepal.

Shape of style.—Curved twice.

Color of stigma.—Pale yellow.

Shape of ovary.—Slightly arched.

Basic color of ovary.—Greenish

Pigmentation of the ovary.—Without red/purple.

Dominant color of male flower.—Cream.

Irregularflowers.—Absent.

Arrangement of ovules.—Not developed.

Characteristics of the Fruit:

Position offruit.—Curved upwards.

Number offruits in the middle hand - More than 14.

Length offruit.—Medium.

Shape offruit.—Curved.

Transverse section of fruit.—Slightly ridged

Apex of fruit.—Nipple-shaped.

Remains offlower parts at apex offruit.—Few floral remains.

Length ofpedicel of fruit.—Long.

Width of pedicel of fruit.—Medium.

Hairiness of pedicel.—Not hairy.

Fusion of pedicels.—Not fused.

Color of skin after maturity.—Green.

Thickness of skin.—Medium.

Adherence of the skin.—Fruit peels easily.

Presence of cracks in skin.—Rarely.

Presence ofpulp.—Yes.

Color of the pulp before maturity.—White.

Color of the pulp after maturity.—Cream

Fruit falling before maturity.—No.

Fruit is eaten.—Cooked green.

Texture ofpulp.—Soft.

Taste.—Mild.

Main use.—Cooking banana.

Presence of seeds with source of pollen.—Few.

Appearance of seeds.—Slightly wrinkled

Shape of seeds.—Rounded.

Agronomic Characteristics (Averages Taken During 1998-1999 for First Crop in a Plot of 10 Plants with No Control of Diseases):

Days from planting to firstflowering.—325.

Number of leaves atflowering.—13.

Number offunctional leaves (less than 15% of area necrotic) atflowering.—13.

Number of leaves at harvest.—10.

Number offunctional leaves (less than 15% of area necrotic at harvest.—9.

Days from flowering until harvest.—105.

Bunch weight.—140.0 kg.

Number of hands.—12.5.

Finger length.—14.1 cm.

Number offingers per bunch.—214.

Daysfrom first flowering until secondflowering.—250.

This cooking banana plant and its fruit described above may vary slightly in detail due to cultural practices, soil types and climatic conditions under which the variety may be grown; the present description is that of the variety grown under the ecological conditions prevailing on the FHIA experimental station near La Lima, Cortes, Honduras.

1. A new and distinct variety of cooking banana plant, substantially as illustrated and described, which is a dwarf plant and has a high level of resistance to the black Sigatoka leaf spot disease; the green fruit is further characterized by having a very good flavor and texture when boiled, to having a long green life after harvest, and to being easy to peel for cooking.
